(19) United States
(12) Patent Application Publication
(10) Pub. No.: US 2018/0226151 A1
(43) Pub. Date: Aug. 9, 2018

(54) SYSTEM AND METHOD FOR IMPROVING FLOAT POOL NURSE SCHEDULING

(71) Applicant: KONINKLIJKE PHILIPS N.V., EINDHOVEN (NL)

(72) Inventors: Reza SHARIFI SEDEH, Malden, MA (US); Yang YANG, Medford, MA (US); Wei WANG, Somerville, MA (US); Yugang JIA, Winchester, MA (US)

(21) Appl. No.: 15/884,867

(22) Filed: Jan. 31, 2018

Related U.S. Application Data
(60) Provisional application No. 62/454,077, filed on Feb. 3, 2017.

(51) Int. Cl. G16H 40/20; G06Q 10/10

(52) U.S. Cl. CPC G16H 40/20; G06Q 10/1093

(57) ABSTRACT

The present disclosure pertains to a system, a method of using such a system, and a non-transitory computer-readable medium containing instructions to such a system to assist in scheduling of float pool nurses. The system, method, and computer-readable medium allow a healthcare facility to optimize float pool nurse scheduling by identifying those units within the healthcare facility that have negatively correlated variation in nurse demand compared to the projected average nurse demand and solicit an approval from a decision-making authority, so that the identified and approved unit pairs can be combined when scheduling float pool nurses for the units.

### CROSS-REFERENCE TO PRIOR APPLICATIONS

This application claims the benefit of U.S. Patent Application No. 62/454,077, filed on Feb. 3, 2017. This application is hereby incorporated by reference herein.

### FIELD OF INVENTION

The present invention relates generally to the field of computer-facilitated scheduling of healthcare staff, and more specifically, to computer-facilitated scheduling of float pool nurses for a healthcare facility that contains multiple units.

### BACKGROUND

A healthcare facility must ensure that a sufficient number of healthcare staff is on duty to provide adequate medical care to patients under the healthcare facility's care. In a typical healthcare facility, nurses are one of the main constituents of healthcare staff providing medical care to patients. A healthcare facility may also be comprised of two or more units (e.g., clinical units) that admit patients and provide for medical care. Thus, staffing an adequate number of nurses to meet the nurse demand of each unit of a healthcare facility is critical to the operation of the healthcare facility. The total number of nurses needed to meet the patient demand may be proportional to the number of patients that are under the care at a unit at a given time.

A unit of a healthcare facility may experience a fluctuating number of patients over time that creates a variable nurse demand over time. For example, a primary care unit (e.g., family medicine unit) of a healthcare facility may experience a higher number of patients under its care and a correspondingly higher nurse demand during day times compared to night times. As another example, a sleep clinic unit of a healthcare facility may experience a higher number of patients under its care and a correspondingly higher nurse demand during the late evenings and early mornings compared to early afternoons. To ensure that an adequate level of medical care is available for patients, a nurse scheduling manager of a healthcare facility takes into account anticipated nurse demand when determining the work schedule of its core nurses (e.g., regularly staffed nurses).

A nurse scheduling manager of a healthcare facility may use a computer system or a computer-facilitated method when scheduling core nurses (e.g., regularly staffed nurses). For example, a computer system may facilitate and/or implement projection of future nurse demand for a healthcare facility (or a unit of a healthcare facility) based on historical patient data to suggest nurse staffing level that may provide adequate levels of medical service to patients.

The actual number of patients under care in a given unit may often be different than the projected number of patients. Hence, the actual nurse demand at a healthcare facility at a given time may be different (i.e., higher or lower) than the projected demand that the nurse scheduling manager relied on to schedule the core nursing staff. If a higher-than-projected number of patients are under a unit's care, then an excess nurse demand may exist (i.e., a unit may have too few nurses to meet the nurse demand of the patients). To meet this excess nurse demand, a nurse scheduling manager may hire and/or schedule other nurses in addition to the core nurses already scheduled, so that the additional nurses can be assigned to units experiencing higher than projected level of nurse demand. The additional nurses may be hired from various non-core staff sources (e.g., nurse staffing agencies, nurse registries, zero-hour nurse staff, or float pool nurses). A healthcare facility may hire float pool nurses to meet the excess nurse demand since float pool nurses may be less expensive to hire compared to nurses from other non-core staff sources. In addition, float pool nurses are shared between different clinical units, thereby providing a flexible option for healthcare facilities in coping with excess nurse demand when the core nurse staffing level is inadequate.

However, labor cost (e.g., per-hour wage) for float pool nurses are often significantly higher than the labor cost for core-nurses (e.g., regularly staffed nurses). Thus, relying on float pool nurses to meet fluctuating nurse demand may pose a significant financial burden to a healthcare facility. In addition, hiring too many float pool nurses results in idling of nurse staff, which is an inefficient use of the healthcare facility's human resource. On the other hand, not hiring enough float pool nurse results in a shortage of nurse staff, leading to patients not receiving adequate medical care and/or nurses experiencing increased stress and diminished work satisfaction.

Accordingly, there is a need for a system and/or a method for scheduling float pool nurses with improved efficiency to minimize the total float pool nurses scheduled for a healthcare facility while maintaining an adequate number of nurse staff on duty to meet the nurse demand of each unit of the healthcare facility.

### SUMMARY

In summary, one aspect of the invention provides a computer-facilitated method for improving float pool nurse staffing, the method comprising: accessing, using a data retrieval module executable by a processor, historical patient data of each unit of a healthcare facility comprising a plurality of units, the data being stored in an electronic memory device; calculating, using a demand calculation module executable by a processor, nurse demand variations for each unit in a healthcare facility from the historical patient data; calculating, using a clustering module executable by a processor, correlations of nurse demand variations between each paired units of all possible unit pairs, identifying, using a clustering module executable by a processor, one or more unit pair suggestions having sufficient negative correlations of the nurse demand variations; soliciting, using an input soliciting module executable by a processor, an input from a decision-making authority, the input comprising information on acceptability of the one or more unit pair suggestions as unit pair recommendations, the unit pair recommendations being those unit pair suggestions that satisfy at least one clustering criteria; and providing, using an output module executable by a processor, an output comprising the one or more unit pair recommendations, wherein the modules are configured together to provide the output that is used to a combined float pool nurse schedule for each unit pair recommendations.

Another aspect of the invention provides a system for improving float pool nurse staffing, comprising: one or more computer processors; and machine-readable instruction modules which, when executed by the one or more processors, cause one or more processors to: using a data retrieval instruction module, access historical patient data of each unit of a healthcare facility comprising a plurality of units, the data being stored in an electronic memory device; using a demand calculation instruction module, calculate nurse demand variations for each unit in a healthcare facility from the historical patient data; using a clustering instruction module, calculate correlations of nurse demand variations between each paired units of all possible unit pairs; using a clustering instruction module, identify one or more unit pair suggestions having sufficient negative correlations of the nurse demand variations; using an input soliciting instruction module, solicit an input from a decision-making authority, the input comprising information on acceptability of the one or more unit pair suggestions as unit pair recommendations, the unit pair recommendations being those unit pair suggestions that satisfy at least one clustering criteria; and using an output instruction module, provide an output comprising the one or more unit pair recommendations, wherein the instruction modules are configured together to provide the output that is used to combine variations in nurse demand for each of the one or more unit pair recommendations to schedule a combined float pool nurse schedule for each of the one or more unit pair recommendations.

Yet another aspect of the invention provides a non-transitory computer-readable medium comprising instructions which, when implemented by one or more computers, cause the one or more computers to: access, from an electronic memory device, historical patient-census data of each unit of a healthcare facility comprising a plurality of units; calculate, using a processor, a nurse demand variation for each unit; identify, using a processor, one or more pairs of units having negatively correlated nurse demand variations; identify, using an input from a decision-making authority, one or more acceptable pairs of units having negatively correlated nurse demand variations that satisfy at least one clustering criteria; and providing, using a computing device, a list comprising the one or more acceptable pairs of units having the negatively correlated nurse demand variations to the user, wherein the list provided is used to schedule a combined float pool nurse schedule for each of the pairs of units in the list.

### BRIEF DESCRIPTION OF THE DRAWINGS

FIG. 1 illustrates a system for scheduling float pool nurse staffing, according to invention principles.

FIG. 2 illustrates a method for scheduling float pool nurse staffing, according to invention principles.

FIGS. 3A-3C illustrate steps of calculating the nurse demand variations for each clinical unit, according to invention principles.

FIGS. 4A-4B illustrate examples of presenting information regarding unit pairs having negative correlations of a nurse demand variations, according to invention principles.

### DETAILED DESCRIPTION

A nurse scheduling manager of a healthcare facility may use a computer system and/or a computer-facilitated method to project future nurse demand for each unit within the healthcare facility to plan core nurse schedules. As an example, core nurses may include nurses who have regularly scheduled work hours (e.g., 9 am to 5 pm, Monday to Friday except holidays), work a required number of hours of work in a time period (e.g., three 12-hour shifts per week), and/or are permanent nurse staff at the healthcare facility and/or its units. The nurse schedule manager may also hire additional nurses who can be deployed to different units to meet variations and/or fluctuations in nurse demand among different units. As an example, the additional nurses may include nurses who are hired to supplement core nurses and/or cover fluctuations in nurse demand on a temporary basis, hired through a nurse registry, a short-term staffing agency (e.g., a temp agency), and/or from nurses with work contracts (e.g., nurses with zero hour contracts or float pool nurses) with the healthcare facility and/or its unit. Float pool nurses may be shared between different clinical units and the labor cost (e.g., per-hour cost) of scheduling float pool nurses is typically higher compared to the labor cost of scheduling core-staff nurses. Therefore, the labor cost associated with hiring float pool nurses may represent a significant expense for a healthcare facility. In addition, over- and understaffing of float pool nurses in a unit result in waste of valuable hospital human resource and inadequate medical care for the patients under the unit's care, respectively. Thus, optimizing float pool nurse hiring and/or scheduling ensures adequate medical care for patients, while reducing labor cost associated with hiring float pool nurses.

There are currently no products that provide a computer-facilitated method of optimizing float pool staff scheduling.

The subject technology may calculate variations between an actual nurse demand and a projected nurse demand for each unit within a healthcare facility to identify unit pairs with sufficiently negatively correlated nurse demand variations. The subject technology may solicit acceptance of those identified units by a decision-making authority based on pre-determined selection criteria and/or the decision-making authority's subjective or objective expertise, then combine those accepted unit pairs for the purpose of scheduling float pool nurses. Since float pool nurses are staffed to meet nurse demand variation from the projected average nurse demand, scheduling float pool nurses based on combined nurse demand variations among unit pairs with negatively correlated variation in nurse demand may reduce overall float pool nurses needed for the healthcare facility. As an example, units A and B of a healthcare facility may have negatively correlated nurse demand variations such that Unit A needs one more nurse than its average nurse demand at 10 am of Monday and one less at 10 am of Wednesday, while Unit B needs one less nurse than its average nurse demand at 10 am of Monday and one more at 10 am of Wednesday. In this example, the subject technology may suggest that variation of nurse demand for Units A and B be combined, thereby scheduling one float nurse who can be assigned to both Units A and B so that the nurse can work Monday in Unit A and Wednesday in Unit B.

Advantageously, the subject technology helps a user (e.g., a nurse scheduling manager) to minimize the total number of float pool nurses that needs to be scheduled while maintaining an adequate number of nurse staff on duty to provide medical care for patients. As a result, the subject technology may help a healthcare facility in scheduling adequate number of float pool nurses while reducing expenses related to the labor cost of scheduling float pool nurses.

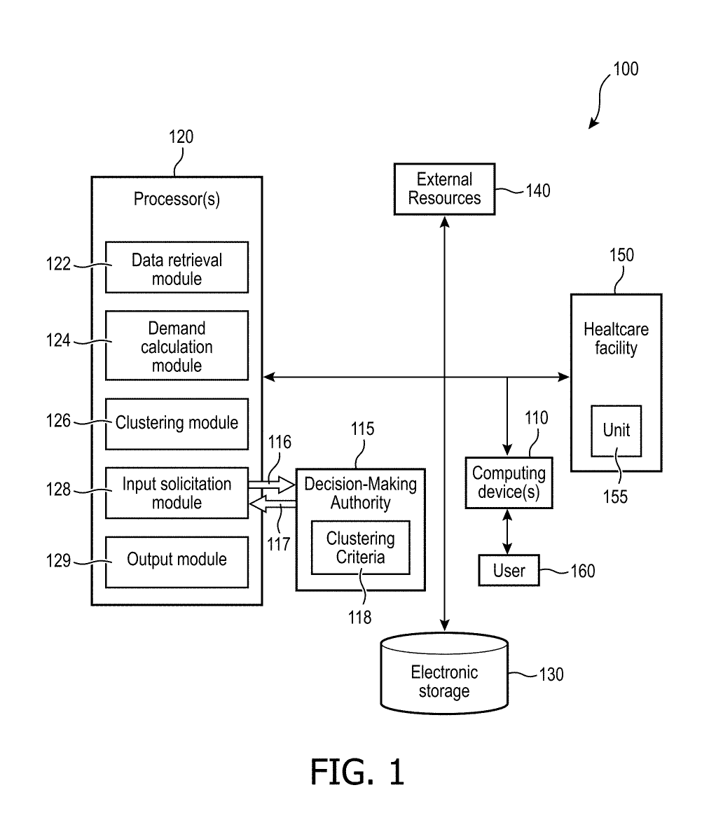

FIG. 1 illustrates a system 100 configured to facilitate nurse scheduling in accordance with one or more embodiments. In some embodiments, system 100 comprises one or more computing devices 110, one or more processors 120, electronic storage 130, external resources 140, and/or other components. Computing devices 110 are configured to provide an interface between user 160 and system 100. In some embodiments, computing devices 110 are associated with healthcare facility 150, unit 155, and/or other entities, service providers associated with or participating in healthcare facility 150 and/or unit 155, and/or other users and/or entities. Computing devices 110 are configured to provide information to user 160, receive information from user 160, and/or solicit an input (e.g., by prompting user 160 to provide an input) from user 160. Computing devices 110 include a user interface and/or other components. The user interface may include a graphical user interface configured to present views and/or fields configured to receive entry and/or selection of patient census information, clustering criteria 118 information, negative correlation threshold information, and/or understaffing/overstaffing threshold information, present information related to unit classifications of units such as unit 155 and/or other units, present computer simulations of patient demand, and/or provide and/or receive other information. In some embodiments, the user interface includes a plurality of separate interfaces associated with, for example, a plurality of computing devices 110, processors 120, and/or other components of system 100. Examples of user 160 include employees, agents, or other persons that may manage and maintain float pool nurse schedule of healthcare facility 150 and/or unit 155, such as a nurse scheduling manager or a hospital administrator.

In some embodiments, one or more computing devices 110 are configured to provide a user interface, processing capabilities, databases, and/or electronic storage to system 100. As such, computing devices 110 may include processors 120, electronic storage 130, external resources 140, and/or other components of system 100. In some embodiments, computing devices 110 are connected to a network (e.g., the Internet). In some embodiments, computing devices 110 do not include processor 120, electronic storage 130, external resources 140, and/or other components of system 100, but instead communicate with these components via the network. The connection to the network may be wireless or wired. For example, processor 120 may be located in a remote server and may wirelessly receive the patient census information from healthcare facility 150 and/or unit 155, and/or cause display of the computer-simulated patient demand via the user interface on a computing device 110 associated with healthcare facility 150 and/or unit 155. In some embodiments, computing devices 110 are laptops, desktop computers, smartphones, tablet computers, smart watches, and/or other computing devices.

Examples of interface devices suitable for inclusion in the user interface include a touch screen, a keypad, touch sensitive and/or physical buttons, switches, a keyboard, knobs, levers, a display, speakers, a microphone, an indicator light, an audible alarm, a printer, and/or other interface devices. The present disclosure also contemplates that computing devices 110 include a removable storage interface. In this example, information may be loaded into computing devices 110 from removable storage (e.g., a smart card, a flash drive, a removable disk) that enables users to customize the implementation of computing devices 110. Other exemplary input devices and techniques adapted for use with computing devices 110 and/or the user interface include, but are not limited to, an RS-232 port, RF link, an IR link, a modem (telephone, cable, etc.) and/or other devices.

In some embodiments, electronic storage 130 comprises electronic storage media that electronically stores information. The electronic storage media of electronic storage 130 may comprise one or both of system storage that is provided integrally (i.e., substantially non-removable) with system 100 and/or removable storage that is removably connectable to system 100 via, for example, a port (e.g., a USB port, a firewire port, etc.) or a drive (e.g., a disk drive, etc.). Electronic storage 130 may be (in whole or in part) a separate component within system 100, or electronic storage 130 may be provided (in whole or in part) integrally with one or more other components of system 100 (e.g., a computing device 110, processor 120, etc.). In some embodiments, electronic storage 130 may be located in a server together with processor 120, in a server that is part of external resources 140, in computing devices 110, and/or in other locations. Electronic storage 130 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 130 may store software algorithms, information obtained and/or determined by processor 120, information received via computing devices 110 and/or other external computing systems, information received from external resources 140, information received from heath care facility 150 and/or unit 155, and/or other information that enables system 100 to function as described herein. By way of a non-limiting example, electronic storage 130 may store the patient census information obtained by data retrieval module 122, the statistical models used by demand calculation module 124, the clustering information determined by clustering module 126, and/or other information.

External resources 140 include sources of information (e.g., databases, websites, etc.), external entities participating with system 100 (e.g., a medical records system of a healthcare facility that stores patient census information), one or more servers outside of system 100, a network (e.g., the Internet), electronic storage, equipment related to Wi-Fi technology, equipment related to Bluetooth® technology, data entry devices, and/or other resources. In some implementations, some or all of the functionality attributed herein to external resources 140 may be provided by resources included in system 100. External resources 140 may be configured to communicate with processor 120, computing device 110, electronic storage 130, healthcare facility 150 and/or unit 155, and/or other components of system 100 via wired and/or wireless connections, via a network (e.g., a local area network and/or the internet), via cellular technology, via Wi-Fi technology, and/or via other resources.

The description and illustration herein (FIG. 1) of a single unit 155 in a single healthcare facility 150 is not intended to be limiting. Healthcare facility 150 may represent any number of healthcare facilities and unit 155 may represent any number of units within any number of healthcare facilities 150. The operations performed by system 100 are applied individually to any number of units 155 in any number of facilities 150. The operations performed by system 100 may be simultaneous for different units 155 and/or performed at different times. For example, system 100 may receive historical patient data for a plurality of units 155 (e.g., from the same healthcare facility 150 and/or from different healthcare facilities 150) and carry out the operations described herein for the plurality of units at the same time. In some embodiments, healthcare facilities 150 include healthcare management systems, hospitals, hospital systems, accountable care organizations, doctor's offices, collections of doctor's offices, and/or other healthcare facilities. Units 155 may include departments within healthcare facilities (e.g., an emergency department, a critical care unit, a neonatal intensive care unit, an imaging department, a laboratory, a surgical department, a maternity department, a pediatric department, a trauma department, a general department, a psychiatric department, a coronary department, and/or other departments), and/or different types of doctor's offices (e.g., family practitioners, pediatricians, orthopedic doctors, cardiologists, oncologists, geriatric doctors, and/or doctors with other medical specialties), and/or other units.

Processor 120 is configured to provide information processing capabilities in system 100. As such, processor 120 may comprise one or more of a digital processor, an analog processor, a digital circuit designed to process information, an analog circuit designed to process information, a state machine, and/or other mechanisms for electronically processing information. Although processor 120 is shown in FIG. 1 as a single entity, this is for illustrative purposes only. In some embodiments, processor 120 may comprise a plurality of processing units. These processing units may be physically located within the same device (e.g., a server), or processor 120 may represent processing functionality of a plurality of devices operating in coordination (e.g., one or more servers, computing devices 110, devices that are part of external resources 140, electronic storage 130, and/or other devices.)

In some embodiments, processor 120, external resources 140, computing devices 110, electronic storage 130, systems that are part of healthcare facility 150 and/or unit 155, and/or other components may be operatively linked via one or more electronic communication links. For example, such electronic communication links may be established, at least in part, via a network such as the Internet, and/or other networks. It will be appreciated that this is not intended to be limiting, and that the scope of this disclosure includes embodiments in which these components may be operatively linked via some other communication media. In some embodiments, processor 120 is configured to communicate with external resources 140, computing devices 110, electronic storage 130, the systems that are part of healthcare facility 150 and/or unit 155, and/or other components according to a client/server architecture, a peer-to-peer architecture, and/or other architectures.

It should be appreciated that although components 122, 124, 126, 128, and 129 are illustrated in FIG. 1 as being co-located within a single processing unit, in embodiments in which processor 120 comprises multiple processing units, one or more of components 122, 124, 126, 128, and/or 129 may be located remotely from the other components. The description of the functionality provided by the different components 122, 124, 126, 128, and/or 129 described below is for illustrative purposes, and is not intended to be limiting, as any of components 122, 124, 126, 128, and/or 129 may provide more or less functionality than is described. For example, one or more of components 122, 124, 126, 128, and/or 129 may be eliminated, and some or all of its functionality may be provided by other components 122, 124, 126, 128, and/or 129. As another example, processor 120 may be configured to execute one or more additional components that may perform some or all of the functionality attributed below to one of components 122, 124, 126, 128, and/or 129.

In some embodiments, data retrieval module 122 is configured to access and retrieve historical patient data for each unit 155 (e.g., a clinical unit of a healthcare facility). As an example, the historical patient data may be historical patient census data that comprise of information regarding patient admission to unit 155, discharge from unit 155, and transfer to and from unit 155. In some embodiments, the historical patient census data may indicate one or more quantities of patient visits to the individual unit with respect to one or more past time periods (e.g., past weeks or other past time periods). As an example, a quantity of patient visits to unit during past periods of time may comprise an hourly (and/or other time-based metric) quantity of patient visits to unit 155 over the past time periods.

In some embodiments, historical patient data is a part of data typically recorded via computing devices 110 and/or other electronic systems associated with unit 155 and/or healthcare facility 150. For example, unit 155 may electronically record when a patient visits unit 155 for an appointment and/or for other reasons (e.g., an emergency or transfer from another unit) via a computing device 110 operated by a staff member of unit 155. The historical patient census data may include recordings of a series of such visits over time (minutes, hours, days, weeks, months, years) by any number of individual patients to unit 155. In some embodiments, data retrieval module 122 may be configured to access information from servers and/or other databases associated with healthcare facility 150 and/or unit 155, servers and/or databases included in external resources 140, electronic storage 130 and/or from other sources.

In some embodiments, data retrieval module 122 is configured to impute missing data and remove outliers from the accessed historical patient data. In addition, data retrieval module 122 may be further configured to remove data from the obtained historical patient data to account for irregularities (such as holidays, holiday weeks, and/or doctors' vacations), and/or to perform other data pre-processing operations. The imputation of missing data and removal of outliers from the obtained historical patient data may be performed with standard imputation algorithms and/or outlier detection procedures, and/or other techniques. As an example, historical patient data for holidays and/or holiday weeks may be removed from the obtained historical patient data because holidays and doctors' vacations create turbulences in patient data patterns and/or for other reasons (e.g., a holiday that occurs on Monday may effectively push Monday patient visits in unit 155 to Tuesday).

In some embodiments, data retrieval module 122 is configured to receive from user 160 via a user interface of computing device 110 as input, data relating to holidays and other special events. Data retrieval module 122 may use such inputs to modify obtained historical patient data (e.g., to better align the demand data with expected demand levels in the future). In some embodiments, data retrieval module 122 may provide instructions to demand calculation module 124 with respect to various days in one or more time periods as blocked out days such that service providers are not allocated to work during those blocked out days.

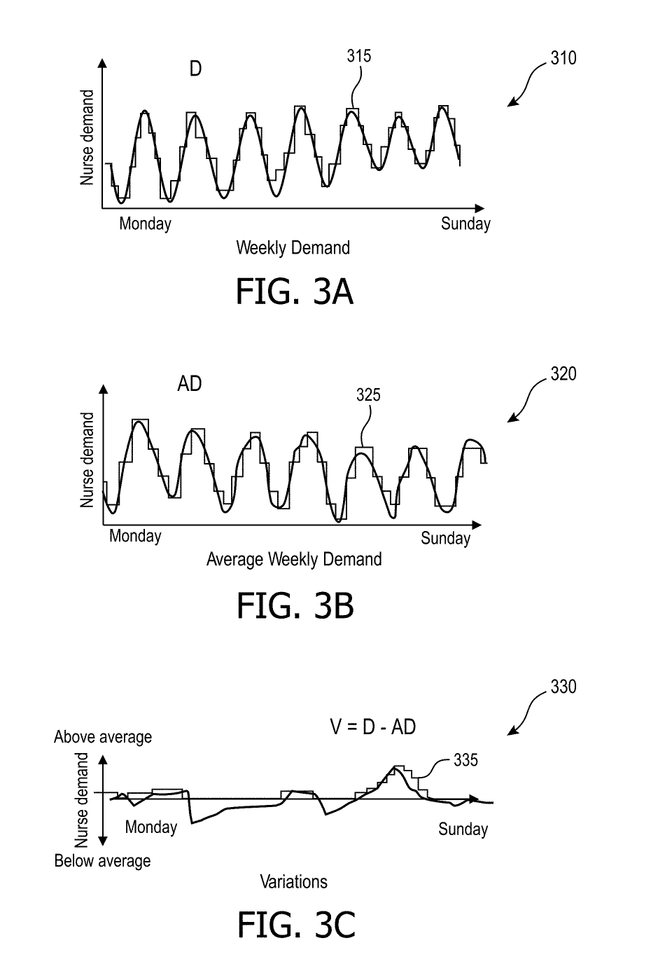

In some embodiments, demand calculation module 124 is configured to determine variation in nurse demand for unit 155 through a series of calculations starting with historical patient data. As shown in FIG. 3, weekly nurse demand 310 of one or more past years can be determined based on historical patient data, average weekly nurse demand 320 can be determined from two or more weekly nurse demand 310, and variations in each of the two or more weekly nurse demand 330 can be determined by subtracting the average weekly nurse demand 320 from each of the weekly nurse demand 310.

In some embodiments, demand calculation module 124 may calculate weekly nurse demand from historical patient data using an optimal ratio between patients and nurses required for unit 155. As an example, the optimal patient to nurse ratio (e.g., number of patients:number of nurses for a given unit) may be between 10:1, 9:1, 8:1, 7:1, 6:1, 5:1, 4:1, 3:1, 2:1, 1:1, and/or other appropriate ratio. In some embodiments, the optimal ratio between patient and nurses may be a ratio or a set of ratios prescribed by a regulation or law of a jurisdiction of unit 155 and/or of healthcare facility 150. In some embodiments, the optimal ratio between patient and nurses may be a predetermined ratio or set of ratios prescribed and/or recommended by an industry or a professional organization (e.g., American Nurses Association). In some embodiments, the optimal ratio between patient and nurses may be determined at the discretion of user 160, management of healthcare facility 150 and/or unit 155, a consultant, or other employees or agents of healthcare facility 150 and/or unit 155.

In some embodiments, demand calculation module 124 may calculate nurse demand to indicate one or more quantities of nurse that are needed for unit 155 with respect to one or more past time periods (e.g., past weeks or other past time periods). As an example, demand calculation module 124 may calculate levels of nurse demand comprising hourly and/or consecutive four-hour increments and/or other time-based metric over a past time period (e.g., daily, weekly, and/or other time periods).

In some embodiments, demand calculation module 124 may calculate average weekly nurse demand in increments of consecutive four-hour periods 325 (or other given periods) to determine the demand information. As an example, the past levels of demand for a unit 155 for a given four-hour period on the first Mondays in February over the last few years may be averaged to determine an estimated level of demand for the given four-hour period for a future first Monday of February. If, for instance, there are four values—f1, f2, f3, and f4—representing the past levels of demand for a unit 155 for the respective four-hour periods, the average of the four values may be calculated by using addition operands to sum the four values and a division operand to divide the sum by four (e.g., (f1+f2+f3+f4)/4).

In some embodiments, demand calculation module 124 may calculate variations in weekly nurse demand 330 for each unit 155 of healthcare facility 150 for each of the time periods (e.g., past weekly time periods) by subtracting average weekly nurse demand 320 from weekly nurse demand 310 for each incremental time unit (e.g., consecutive four-hour periods 315) for each past week, thereby representing variations in weekly nurse demand 330 in increments of consecutive four-hour periods 335.

It should be appreciated that, although some nurse demand calculations illustrated above use quantity of nurses in hourly or consecutive four-hour period increments over past weekly periods, these are for illustrative purposes only and the time increment and time period for calculating and representing nurse demand may include any combination of time increments (e.g., seconds, minutes, hours, days, weeks) over any time period (e.g., day, week, month, year) that may be deemed appropriate.

Referring back to FIG. 1, in some embodiments, system 100 may be configured so that clustering module 126 identifies and clusters unit pairs 155 that have negatively correlated variation between them. In some embodiment, the demand calculated by the demand calculation module for each unit is paired with all possible unit pairs.

In some embodiments, clustering module 126 may calculate variation correlation between all possible unit pairs within a healthcare facility. As an example, in a healthcare facility consisting of Units A, B, C, D, and E, the all possible unit pairs include: Units A and B; Units A and C, Units A and D, Units A and E; Units B and C; Units B and D; Units B and E; Units C and D; Units C and E; and Units D and E. In another embodiment, clustering module 126 may calculate variation correlation between some, but not all units within healthcare facility 150 by using one or more criteria to exclude certain unit pairs (e.g., user input, pre-defined list of units not to be paired, or other similar criteria).

In some embodiments, clustering module may calculate variation correlations between each of the possible unit pairs using Pearson product-moment correlation coefficient. In other embodiments, variation correlations between units may be determined using Spearman's correlation or any other well-known ways of calculating correlation.

In some embodiments, clustering module 126 may calculate correlations of nurse demand variations between unit pairs to indicate one or more quantities of correlation (e.g., correlation coefficient) with respect to one or more past time periods (e.g., past weeks or other past time periods). As an example, correlation of nurse demand variation between paired units may be calculated based on hourly (and/or other time-based metric) nurse demand variation over the past time periods (e.g., daily, weekly, and/or other time periods).

In some embodiments, clustering module 126 may identify and select those unit pairs that have correlation coefficient values (e.g., Pearson coefficient) that exceed a threshold value (e.g., more negative than −0.7) or unit pairs that have correlation coefficient values that are within a predetermined range (e.g., between −0.7 and −1.0). In some embodiments, the threshold correlation value may be determined using other metrics (e.g., ranking pairs by their correlation coefficient values). In some embodiments, a threshold correlation coefficient or a range can be set and adjusted by a user.

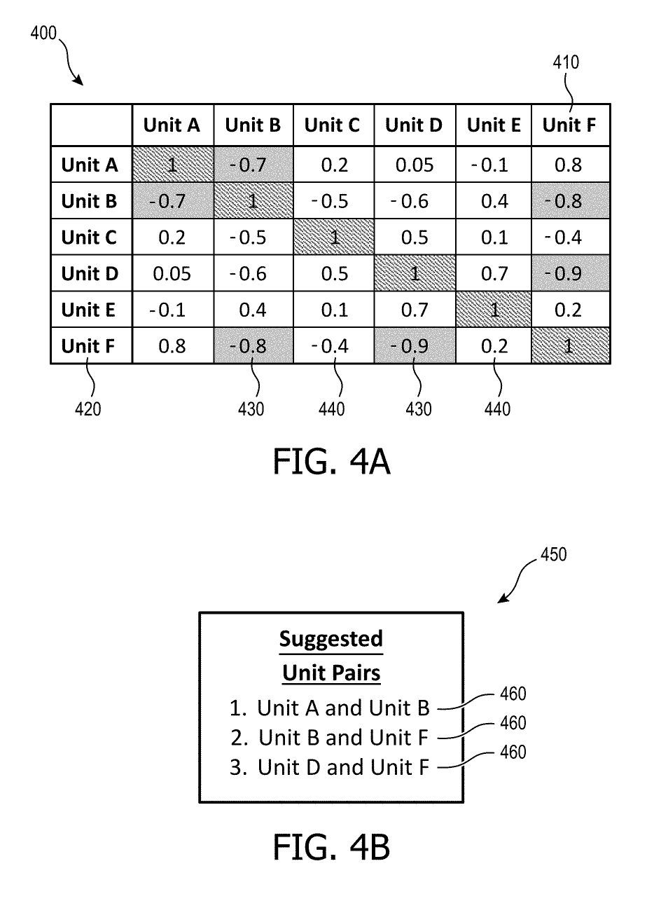

In some embodiments, clustering module 126 may identify a plurality of pairs wherein a common single unit is a constituent in two or more pairs. As an example, as shown in FIGS. 4A and B, clustering module 126 may identify (indicated by shading) unit pairs A and B, and also identify unit pairs B and F based on their negative correlation of nurse demand variation. In such an instance, clustering module 126 may keep one or more unit pairs with highest degree of negative correlation of nurse demand variation. As an example, as shown in FIG. 4A, the unit pair consisting of units A and B has a correlation coefficient (e.g., Pearson coefficient) of −0.7 whereas the unit pair consisting of units B and F has a correlation coefficient of −0.8. In such an instance, clustering module 126 may eliminate the pair with less negative correlation (i.e., unit pairs A and B). In another example, input solicitation module may determine by other appropriate criteria (e.g., predetermined criteria, user input, and/or other appropriate criteria) whether to include or exclude one or more of plurality of unit pairs that have a common single unit as a constituent.

In some embodiments, after clustering module 126 has identified one or more pairs with negative variation coefficient, input solicitation module 128 may make unit pair suggestions 116 to decision-making authority 115 and solicit input 117 to determine whether the unit pair suggestions satisfy one or more acceptable clustering criteria 118.

In some embodiments, the input solicitation module 128 solicits input 117 from decision-making authority 115 (e.g., nurse scheduling manager) by providing a suggestion 116 (e.g., printout, screen prompt, voice prompt, text message, email, and/or other types of output) via computing device 110 that prompts user 160 to assess suggested negatively correlated unit pairs based on at least one clustering criteria 118. In response, user 160 can make input 117 (e.g., accept or reject each of the proposed unit pairs) using an interface devices (e.g., keyboard input, mouse, touch screen, lever, and/or other types of inputting device) of computing device 110 indicating whether each of the proposed pairs are acceptable based on at least one clustering criteria 118. As an example, clustering criteria 118 used to determine whether a unit pair is appropriate for clustering may be based on: (1) a user's expertise (e.g., experience and intuition); (2) data collected from nurse talent card; (3) preferences of individual nurses for working in certain units, geographical limitations (e.g., distance between the nurse's home and the location of the clinical unit and/or distance between the units that are paired); (4) nurse skill set; and (5) other criteria that a user may use, or can be predetermined.

As shown in FIG. 4A, in some embodiments, input solicitation module 128 may provide suggestion 116 comprising all nurse demand variation correlation information, (e.g., both recommended unit pairs 430 and unit pairs that are not recommended 440) for all possible unit pairs by providing a table 400, wherein each unit is paired with every unit by having column headings 410 and row headings 420 that comprise every unit of a healthcare facility. In such embodiments, the suggested unit pairs may be indicated by shading 430 or by other visual cues.

As yet another example, input solicitation module 128 may provide suggestion 116 to decision-making authority 115 comprising recommended pairs of clinical units where a list of unit pair suggestions is presented in a text format 450, as shown in FIG. 4B. Such suggestion may comprise suggested pair units 460 that are represented in a list format.

In some embodiments, decision-making authority 115 may be another computer algorithm, configured to either accept or reject suggestion 116 based on one or more acceptable clustering criteria 118 and provide input 117 to the input solicitation module 128.

In some embodiments, decision-making authority 115 may comprise both user 160 and a computer algorithm. For example, a computer algorithm decision-making authority may make a preliminary determination on the acceptability of unit pair suggestions and a final determination on the acceptability of unit pair suggestions and input 117 are made by user 160. Further, in another example, a user decision-making authority 115 may make the preliminary determination and a computer algorithm may make the final determination to accept or reject suggested unit pairs and provide input 117 to input solicitation module 128.

In some embodiments, input solicitation module 128 may generate a unit pair recommendation by revising unit pairs suggestions 116 based on input 117 by decision-making authority 115. For example, input solicitation module 128 may remove those unit pairs that were rejected by input 117, and keep those unit pairs that are accepted by input 117 to generate a list of recommended unit pairs.

It should be appreciated that although decision-making authority 115 is shown outside of processor 120, computing device 110, electronic storage 130, external resources 140, healthcare facility 150, and unit 155, decision-making authority 115 may be processed by processor 120 (e.g., as an algorithm decision-making authority), stored in electronic storage 130, stored in external resources 140, and/or may use computing device 110 to provide input 117. In addition, decision-making authority 115 may also be user 160 who is associated with healthcare facility 150 and/or unit 155 (e.g., a nurse scheduling manager of a healthcare facility and/or a unit) and provide input 117 via computing device 110 (e.g., using interface device within computing device 110).

In some embodiments, output module 129 is configured to provide an output comprising recommended unit pairs for clustering nurse demand variation data. In some embodiments, the recommended unit pairs are identified by clustering module 126 as having acceptable negative nurse variation correlation between the paired units and/or determined to be acceptable by input solicitation module 128.

In some embodiments, the output module 129 may provide an output using computing devices 110 (e.g., monitor/screen display, print out, and/or other visual output) that a user (e.g., a nurse scheduling manager) can use for determining future float pool nurse schedules. As another example, output module 129 may provide an output in the form of a telecommunications message (e.g., text message, emails, automated telephone voice call, computer-generated voice mail, and/or other telecommunications message) that may be used to schedule float pool nurses and/or provide instructions for float pool nurses regarding their individual schedules. As another example, output module 129 may provide an output in the form of digital data signal or other input data appropriate for instructing another system and/or algorithm to effectuate computer-facilitated scheduling or updating of a nurse schedule calendar.

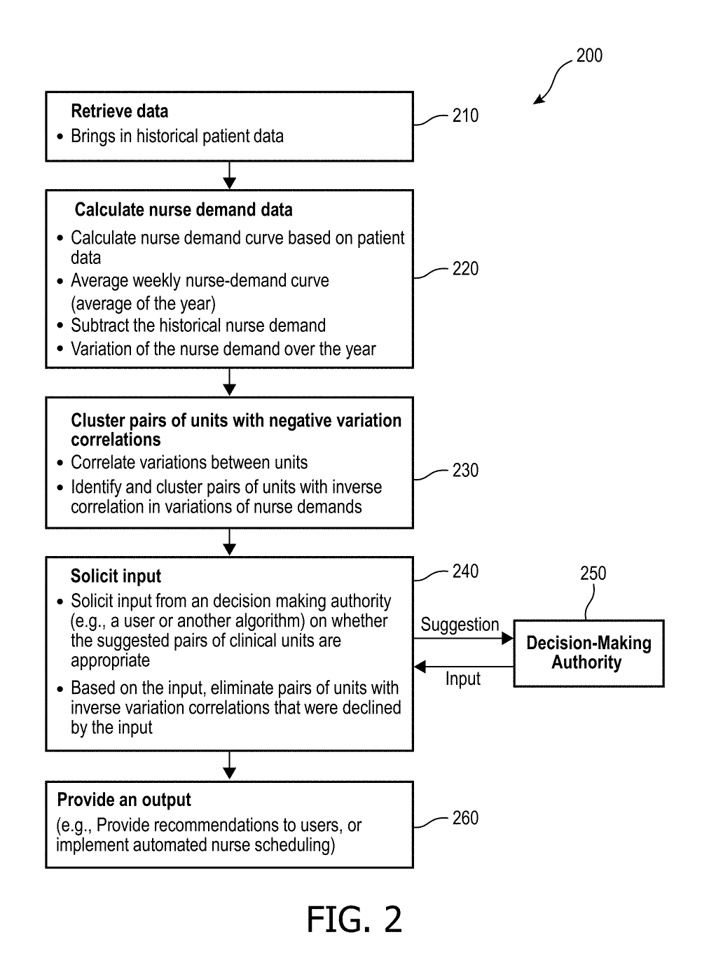

FIG. 2 is a flow diagram of a method 200 that may be used by embodiments described herein for improving float pool nurse scheduling. Method 200 may be performed by a float pool nurse scheduling system (e.g., system 100). Several non-limiting embodiments of the system 100 that may use method 200 is described elsewhere in this application. The operations of method 200 presented below are intended to be illustrative.

At an operation 210, historical patient data for an individual unit of a healthcare facility may be accessed and/or retrieved. As an example, historical patient data may be accessed from servers and/or other databases associated with healthcare facility 150 and/or unit 155, servers and/or databases included in external resources 140, electronic storage 130, computing devices 110, and/or from other sources.

In some embodiments with respect to operation 210, outliers such as holidays that cause historical patient data irregularities are removed from the accessed historical patient data. In other embodiments with respect to operation 210, missing data is imputed, for example, using standard imputation algorithms and/or other techniques. In some embodiments with respect to operation 210, holidays and other special events may be obtained from user input and may modify accessed and/or retrieved historical patient data.

In some embodiments, operation 210 is performed by a processor component that is the same as or similar to data retrieval module 122 (shown in FIG. 1 and described herein).

At an operation 220, various nurse demand data may be calculated from historical patient data. In some embodiments with respect to operation 220, nurse demand calculation includes steps of compiling nurse demand data for different units based on historic patient data (e.g., patient census data). In some embodiments, the nurse demand may be calculated based on an optimal patient to nurse ratio.

In some embodiments with respect to operation 220, average weekly nurse demand 320 may be determined by calculating the average of plurality of weekly nurse demand 310 for one or more past time periods (e.g., past weeks or past time periods).

In some embodiments with respect to operation 220, variation in nurse demand 330 may be determined by subtracting the average weekly nurse demand 320 values from weekly nurse demand 310 for each of the units 155 for past time periods (e.g., weekly, monthly, and/or another time period), as shown in FIGS. 3A-C. In some embodiments, operation 220 is performed by a processor component that is the same as or similar to demand calculation module 124 (shown in FIG. 1 and described herein).

At an operation 230, unit pairs with sufficient negatively correlated nurse demand variations may be identified. As an example, correlations of nurse demand variation between all possible unit pairs may be calculated. In some embodiments, unit pairs with sufficient negatively correlated nurse demand variations may be determined by calculating correlation of nurse demand variations between paired units to indicate one or more quantities of correlation (e.g., correlation coefficient) for a unit with respect to one or more past time periods (e.g., past weeks or other past time periods). In some embodiments with respect to operation 230, correlations between the unit pairs may be determined by calculating Pearson product-moment correlation coefficient for each of the unit pairs. In other embodiments with respect to 230, other well-known ways of calculating correlation may be used to determine correlation of variations between each of the unit pairs. In some embodiments with respect to 230, those unit pairs that have correlation coefficient values (e.g., Pearson coefficient) that exceed a threshold value (e.g., more negative than a preset negative correlation coefficient value such as −0.7) or units that have correlation coefficient values that are within a predetermined range (e.g., between −0.7 and −1.0) are identified and selected. In some embodiments, the criteria for selection is determined using other metrics (e.g., by ranking unit pairs by their correlation coefficient) and/or the criteria for selection can be set and adjusted by a user. In some embodiments, operation 230 is performed by a processor component that is the same as or similar to clustering module 126 (shown in FIG. 1 and described herein).

At an operation 240, the unit pairs that are identified in operation 230 may be provided as suggestions to decision-making authority 250 and an input may be solicited from decision-making authority 250 to determine whether or not the suggested unit pairs meet one or more clustering criteria 118 to be acceptable for clustering. In some embodiments with respect to operation 240, unit pair suggestions are presented to decision-making authority 250 (e.g., nurse scheduling manager) by, for example, providing an output (e.g., printout, screen prompt, voice prompt, text message, email, and/or other types of output) via computing device 110 and prompting user 160 to assess proposed negatively-correlated unit pairs based on at least one clustering criteria 118. In some embodiments with respect to operation 240, unit pair suggestions may comprise nurse demand correlation information for all possible unit pairs calculated in operation 230 with the suggested pairs being distinguished visually (e.g., by shading) as shown in FIG. 4A. In other embodiments with respect to operation 240, an output comprises a list of suggested unit pairs that were identified by operation 230 as shown in FIG. 4B. In response, an input from decision-making authority 250 (e.g., user 160) may be received via an interface device (e.g., keyboard input, mouse, touch screen, lever, and/or other types of inputting device) of computing device 110 indicating whether each of the suggested unit pairs are acceptable based on at least one clustering criteria 118. In another embodiment with respect to operation 240, an input is solicited from a decision-making authority 250 that is a computer algorithm configured to either accept or reject the proposed unit pairs based on at least one clustering criteria 118. In some embodiments with respect to operation 240, the suggested unit pairs identified from operation 230 is revised based on input from decision-making authority 250 (e.g., one or more unit pairs that were not accepted by decision-making authority 250 are removed and/or keep the one or more unit pairs that were accepted by the decision-making authority 250). In some embodiments, operation 240 is performed by a processor component that is the same as or similar to input solicitation module 126 (shown in FIG. 1 and described herein) and may use computer device 110, external resources 140, electronic storage 130, and/or processor 120 to solicit and receive input from decision-making authority 250.

At an operation 260, an output comprising recommended unit pairs with negative nurse demand correlation that were accepted by decision-making authority 250 is generated and presented to a user. In some embodiments with respect to operation 260, an output is provided in the form of a visual output (e.g., monitor/screen display, print out, and/or other visual output format) that user 160 (e.g., a nurse scheduling manager) uses in determining future float pool nurse schedules. In other embodiments, the output is provided in the form of a telecommunications message (e.g., text message, emails, automated telephone voice call, computer-generated voice mail, and/or other telecommunications message) that may be used to schedule float pool nurses and/or provide instructions for float pool nurses regarding their individual schedules. Such telecommunications message may send messages to computing device 110 and/or to external resources 140 (e.g., individual nurses' smart phones and/or other external computing devices) In other embodiments, the output is provided in the form of digital data signal or other input data appropriate for instructing another system and/or a computer algorithm to effectuate computer-facilitated scheduling or updating of a nurse schedule calendar. In some embodiments, operation 260 is performed by a processor component that is the same as or similar to output module 129 (shown in FIG. 1 and described herein) and an output may be performed via computing device 110.

What is claimed is:

1. A computer-facilitated method for improving float pool nurse staffing, the method comprising:
accessing, using a data retrieval module executable by a processor, historical patient data of each unit of a healthcare facility comprising a plurality of units, the data being stored in an electronic memory device;
calculating, using a demand calculation module executable by a processor, nurse demand variations for each unit in a healthcare facility from the historical patient data;
calculating, using a clustering module executable by a processor, correlations of nurse demand variations between each paired units of all possible unit pairs,
identifying, using a clustering module executable by a processor, one or more unit pair suggestions having sufficient negative correlations of the nurse demand variations;
soliciting, using an input soliciting module executable by a processor, an input from a decision-making authority, the input comprising information on acceptability of the one or more unit pair suggestions as unit pair recommendations, the unit pair recommendations being those unit pair suggestions that satisfy at least one clustering criteria; and
providing, using an output module executable by a processor, an output comprising the one or more unit pair recommendations,
wherein the modules are configured together to provide the output that is used to a combined float pool nurse schedule for each unit pair recommendations.

2. The system of claim 1,
wherein the nurse demand variations for each unit is calculated by steps comprising:
calculating a weekly nurse demand for each week of one or more past years;
calculating an average weekly nurse demand from the weekly nurse demand; and
subtracting the average weekly nurse demand from the weekly nurse demand.

3. The method of claim 1,
wherein the negative correlations are assessed using Pearson product-moment correlation coefficient.

4. The method of claim 1,
wherein the at least one clustering criteria is compatibility of required nurse skill set between constituent units of each unit pair suggestions, geographic proximity of the constituent units of each unit pair suggestions, and/or preference of individual nurse to work in the constituent units of each unit pair suggestions.

5. The method of claim 1,
wherein the decision-making authority is a user.

6. The method of claim 1,
wherein the decision-making authority is an algorithm configured to accept or reject the one or more unit pairs based on one or more predetermined clustering criteria.

7. The method of claim 1,
wherein the output facilitates a user to schedule a combined float pool nurse schedule.

8. The method of claim 1,
wherein the output effectuates a nurse scheduling calendar to be created or adjusted optionally, without a user input.

9. A system for improving float pool nurse staffing, comprising:
one or more computer processors; and
machine-readable instruction modules which, when executed by the one or more processors, cause one or more processors to:
using a data retrieval instruction module, access historical patient data of each unit of a healthcare facility comprising a plurality of units, the data being stored in an electronic memory device;
using a demand calculation instruction module, calculate nurse demand variations for each unit in a healthcare facility from the historical patient data;
using a clustering instruction module, calculate correlations of nurse demand variations between each paired units of all possible unit pairs;
using a clustering instruction module, identify one or more unit pair suggestions having sufficient negative correlations of the nurse demand variations;
using an input soliciting instruction module, solicit an input from a decision-making authority, the input comprising information on acceptability of the one or more unit pair suggestions as unit pair recommendations, the unit pair recommendations being those unit pair suggestions that satisfy at least one clustering criteria; and
using an output instruction module, provide an output comprising the one or more unit pair recommendations,
wherein the instruction modules are configured together to provide the output that is used to combine variations in nurse demand for each of the one or more unit pair recommendations to schedule a combined float pool nurse schedule for each of the one or more unit pair recommendations.

10. The system of claim 9,
wherein the correlations are calculated using Pearson product-moment correlation coefficient.

11. The system of claim 9,
wherein the correlations are calculated using Spearman's correlation coefficient.

12. The system of claim 9
wherein the at least one clustering criteria is compatibility of required nurse skill set between constituent units of each unit pair suggestions, geographic proximity of the constituent units of each unit pair suggestions, and/or preference of individual nurse to work in the constituent units of each unit pair suggestions.

13. The system of claim 9,
wherein the decision-making authority is a user.

14. The system of claim 9,
wherein the decision-making authority is a computer algorithm configured to accept or reject the one or more unit pair suggestions based on the at least one clustering criteria.

15. The system of claim 9,
wherein the output provides a user with an instruction to combine the one or more unit pair recommendations to create a combined float pool nurse schedule.

16. The system of claim 9,
wherein the output schedules a combined float pool nurse schedule by causing a float pool nurse scheduling calendar to be created or adjusted optionally without a user input.

17. A non-transitory computer-readable medium comprising instructions which, when implemented by one or more computers, cause the one or more computers to:
access, from an electronic memory device, historical patient-census data of each unit of a healthcare facility comprising a plurality of units;
calculate, using a processor, a nurse demand variation for each unit;
identify, using a processor, one or more pairs of units having negatively correlated nurse demand variations;
identify, using an input from a decision-making authority, one or more acceptable pairs of units having negatively correlated nurse demand variations that satisfy at least one clustering criteria; and
providing, using a computing device, a list comprising the one or more acceptable pairs of units having the negatively correlated nurse demand variations to the user,
wherein the list provided is used to schedule a combined float pool nurse schedule for each of the pairs of units in the list.

18. The non-transitory computer-readable medium of claim 17,
wherein the negatively correlated nurse demand variations are assessed using Pearson product-moment correlation coefficient.

19. The non-transitory computer-readable medium of claim 17,
wherein the negatively correlated nurse demand variations are assessed using Spearman's correlation coefficient.

20. The non-transitory computer-readable medium of claim 17,
wherein the at least one clustering criteria is compatibility of required nurse skill set between constituent units of each unit pairs, geographic proximity of the constituent units of each pairs of units, and/or preference of individual nurse to work in the constituent units of each pairs of units.

21. The non-transitory computer-readable medium of claim 17,
wherein the decision-making authority is a user.

22. The non-transitory computer-readable medium of claim 17,
wherein the decision-making authority is an algorithm configured to accept or reject the one or more pairs of units that are provided to the decision-making authority based on at least one predetermined clustering criteria.

23. The non-transitory computer-readable medium of claim 17,
wherein the list provided is used to schedule the combined float pool nurse schedule by providing a visual output to a user to create or adjust a combined nurse float pool schedule.

24. The non-transitory computer-readable medium of claim 17,
wherein the list provided is used to schedule the combined float pool nurse schedule by causing a nurse scheduling calendar to be created or adjusted optionally without a user input.

25. The non-transitory computer-readable medium of claim 17,
wherein the list provided is used to schedule the combined float pool nurse schedule by causing a nurse scheduling calendar to be created or adjusted optionally without a user input,
wherein the scheduling calendar causes an automated telecommunication message to be sent to a nurse regarding a schedule of the nurse.
